(19) United States
(12) Patent Application Publication
(10) Pub. No.: US 2020/0122128 A1
(43) Pub. Date: Apr. 23, 2020

(54) METHOD FOR PRODUCING ALIPHATIC LINEAR PRIMARY ALCOHOLS

(71) Applicant: KOREA INSTITUTE OF ENERGY RESEARCH, Daejeon (KR)

(72) Inventors: Min Hye YOUN, Sejong-si (KR); Dong Hyun CHUN, Daejeon (KR); Ji-chan PARK, Daejeon (KR); Heon-do JEONG, Daejeon (KR); Kee-young KOO, Daejeon (KR); Dong-wook LEE, Daejeon (KR)

(21) Appl. No.: 16/579,847

(22) Filed: Sep. 24, 2019

(30) Foreign Application Priority Data
Oct. 19, 2018 (KR) 10-2018-0125563
Sep. 18, 2019 (KR) 10-2019-0114468

(51) Int. Cl. B01J 23/89; B01J 23/75; B01J 37/04; B01J 37/03; B01J 37/06; B01J 37/02; B01J 35/02; C07C 29/16; C07C 1/24

(52) U.S. Cl. CPC B01J 23/8913; B01J 23/75; B01J 37/04; B01J 37/031; C07C 2521/04; B01J 37/0236; B01J 35/026; C07C 29/16; C07C 1/24; B01J 37/06

(57) ABSTRACT

Provided are a method of preparing a linear primary alcohol, a catalyst for converting an α-olefin into an alcohol, and a method of converting an α-olefin into a linear primary alcohol, and the method of preparing a linear primary alcohol according to the present invention includes: charging a reactor with a heterogeneous catalyst including a cobalt oxide and a Cₙ olefin (S1); bringing the heterogeneous catalyst including a cobalt oxide into contact with the Cₙ olefin (S2); and supplying the reactor with a synthetic gas to obtain a Cₙ₊₁ alcohol (S3).

### TECHNICAL FIELD

The following disclosure relates to a method of preparing a linear primary alcohol, a catalyst for converting an α-olefin into an alcohol, and a method of converting an α-olefin into a linear primary alcohol, and more particularly, to a method of preparing a linear primary alcohol having n+1 carbon atoms from a paraffin-olefin mixed fraction having n carbon atoms produced from various chemical byproducts.

### BACKGROUND

(C4-C12) linear α-olefins (LAO) in a certain range of the number of carbon atoms are used as a basic material of various chemical industries such as copolymers of polyolefins, lubricants, and plasticizers, and most of (C4-C12) linear α-olefins are produced in a small amount based on a naphtha cracker or produced by oligomerization of ethylene, but recently, a demand thereof has been increasing steadily. In particular, linear α-olefins having an even number of carbon atoms such as C6, C8, and C10 may be applied as a copolymer of a polyolefin elastomer (POE) and a lube base oil, but since a commercializing process thereof is very limited, production of high-purity linear α-olefins is known as one of the very important techniques in the petrochemical industry.

A conventionally known production process of LAO may be largely classified into the following two processes. A first technique is a method by oligomerization of ethylene, butene, and the like, and is known in KR 1545369 B1, U.S. Pat. No. 4,486,615 A, and the like. Examples of the specific process thereof may include an ethyl corporation process by INEOS Corporation, a gulf process by Chevron Phillips Chemical Company, a SHOP process by Shell Oil Company, a petrochemical process by Idemitsu, an α-sablin process by SABIC-Linde, and the like. A second technique is a method by Fisher-Tropsch, in which a synthetic crude oil having a high olefin content is prepared from synthetic gas and then a linear α-olefin is produced by a separation purification process such as extraction. The commercial process thereof is possessed by Sasol Limited and Exxon mobile Corporation and is known in U.S. Pat. No. 6,787,576 B2 and the like.

Most of the techniques for producing LAO so far focus on ethylene oligomerization, but the unit cost of a raw material in an ethylene oligomerization process is high, so that it is difficult to secure economic feasibility, and it is also difficult to adjust selectivity of an oligomerization reaction, and thus, a problem of additional separation purification is followed. Meanwhile, since a technique of producing LAO from a mixed fraction containing an olefin obtained by the Fisher-Tropsch process of synthetic gas should be performed via a homogeneous or heterogeneous catalyst reaction of three or more steps such as hydroformylation, hydrogenation, and dehydrogenation, separation and purification are difficult and economic feasibility of the process is still insufficient.

### RELATED ART DOCUMENTS

### Patent Documents

KR 1545369 B1
U.S. Pat. No. 4,486,615 A
U.S. 678,757 B2

### SUMMARY

An embodiment of the present invention is directed to providing a method of selectively converting an olefin from a paraffin-olefin mixed fraction having n carbon atoms (Cₙ) produced from various chemical byproducts such as chemical byproducts from Fisher-Tropsch synthesis or a naphtha cracker, into a primary alcohol having n+1 carbon atoms.

Another embodiment of the present invention is directed to providing a method of preparing a Cₙ₊₁ linear primary alcohol economically under a low temperature and low pressure condition by simplifying a conventional two-step process of hydroformylation-hydrogenation into a one-step process of reductive hydroformylation.

Another embodiment of the present invention is directed to providing a method of preparing a high-purity linear α-olefin by conversion into a linear α-olefin having n+1 carbon atoms (Cₙ₊₁) by reductive hydroformylation and subsequent dehydrogenation.

Still another embodiment of the present invention is directed to providing a catalyst for converting an α-olefin into an alcohol having high process efficiency and capable of continuous treatment, by replacing a homogeneous catalyst with a heterogeneous catalyst, and an alcohol conversion method using the same.

In one general aspect, a method of preparing a linear primary alcohol includes charging a reactor with a heterogeneous catalyst including a cobalt oxide and a Cₙ (n is an integer of 4 to 20) olefin (S1); bringing the heterogeneous catalyst including a cobalt oxide into contact with the Cₙ olefin (S2); and supplying the reactor with a synthetic gas to obtain a Cₙ₊₁ alcohol (S3).

According to an exemplary embodiment of the present invention, (S3) includes a reductive hydroformylation reaction of the Cₙ olefin and the synthetic gas.

According to an exemplary embodiment of the present invention, the reductive hydroformylation reaction may be performed at a temperature of 100° C. to 350° C. under a pressure of 15 bar to 60 bar.

According to an exemplary embodiment of the present invention, the cobalt oxide may have a rod shape.

According to an exemplary embodiment of the present invention, a cross section of the rod-shaped cobalt oxide may have an average diameter of 10 to 100 nm and an aspect ratio of 2 to 1000.

According to an exemplary embodiment of the present invention, the heterogeneous catalyst may be one or two or more metals selected from the group consisting of rhodium, palladium, platinum, silver, gold, iridium, ruthenium, and the like supported on cobalt oxide particles.

According to an exemplary embodiment of the present invention, a conversion rate of the olefin in (S3) may be 95% or more.

According to an exemplary embodiment of the present invention, a selectivity of the Cₙ₊₁ alcohol in (S3) may be 40% or more.

According to an exemplary embodiment of the present invention, after (S3), dehydrating the Cₙ₊₁ alcohol to obtain a Cₙ₊₁ olefin (S4) may be further included.

According to an exemplary embodiment of the present invention, (S4) may include the dehydrating step under an alumina catalyst.

In another general aspect, a catalyst for converting an α-olefin into an alcohol includes a heterogeneous catalyst including a cobalt oxide for converting a Cₙ olefin into a Cₙ₊₁ alcohol.

According to an exemplary embodiment of the catalyst for converting an α-olefin into an alcohol of the present invention, the heterogeneous catalyst may be one or two or more metals selected from the group consisting of rhodium, palladium, platinum, silver, gold, iridium, ruthenium, and the like supported on cobalt oxide particles.

According to an exemplary embodiment of the catalyst for converting an α-olefin into an alcohol of the present invention, the cobalt oxide and the supported metal may be included at a weight ratio of 1:0.001 to 1:0.1.

In still another general aspect, a method of converting an olefin into an alcohol includes bringing a mixed fraction including a paraffin and a Cₙ olefin into contact with a heterogeneous catalyst including a cobalt oxide to convert the mixed fraction into a Cₙ₊₁ primary alcohol by a reductive hydroformylation reaction.

Other features and aspects will be apparent from the following detailed description, the drawings, and the claims.

### BRIEF DESCRIPTION OF THE DRAWINGS

FIG. 1 illustrates a process flow chart of a method of preparing a linear primary alcohol according to an exemplary embodiment of the present invention.

FIG. 2 illustrates a process of the method of preparing a linear primary alcohol according to an exemplary embodiment of the present invention.

FIG. 3 illustrates a process flow chart of a method of preparing a linear α-olefin according to an exemplary embodiment of the present invention.

FIG. 4 illustrates a process of the method of preparing a linear α-olefin according to an exemplary embodiment of the present invention.

FIG. 5 illustrates a scanning electron microphotograph (SEM) of a heterogeneous cobalt oxide in a flak shape according to Preparation Example 1.

FIG. 6 illustrates a scanning electron microphotograph (SEM) of a heterogeneous cobalt oxide in a cube shape according to Preparation Example 2.

FIG. 7 illustrates a scanning electron microphotograph (SEM) of a heterogeneous cobalt oxide in a spherical shape according to Preparation Example 3.

FIG. 8 illustrates a scanning electron microphotograph (SEM) of a heterogeneous cobalt oxide in a flat plate shape according to Preparation Example 4.

FIG. 9 illustrates a scanning electron microphotograph (SEM) of a heterogeneous cobalt oxide in an octahedral shape according to Preparation Example 5.

FIG. 10 illustrates a scanning electron microphotograph (SEM) of a heterogeneous cobalt oxide in a rod shape according to Preparation Example 6.

FIG. 11 illustrates conversion rates and selectivities of olefins, as a result of one-step reaction experiments according to Examples 1 to 6 of the present invention.

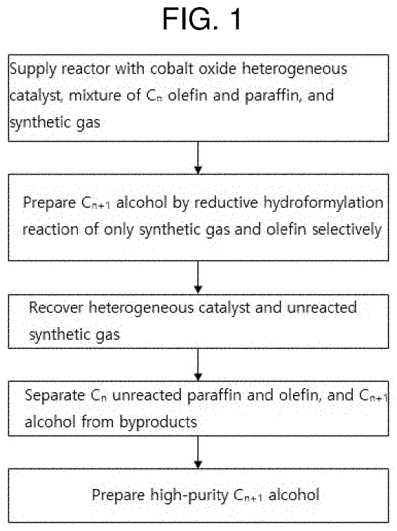

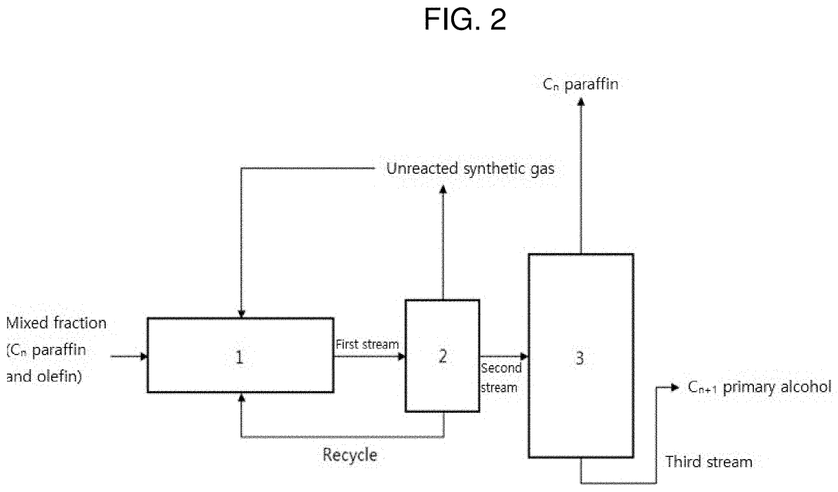

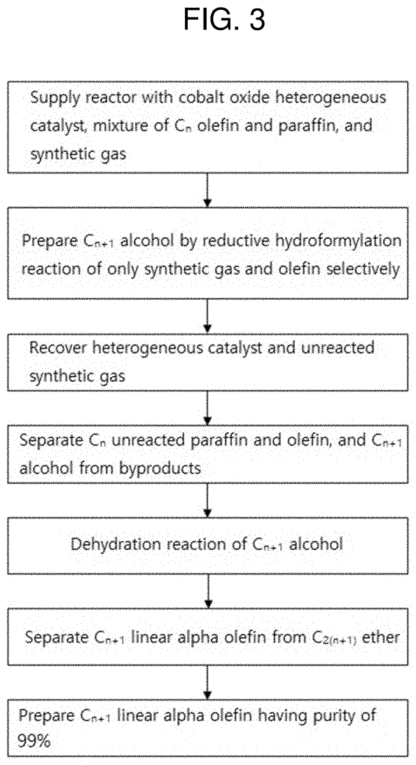

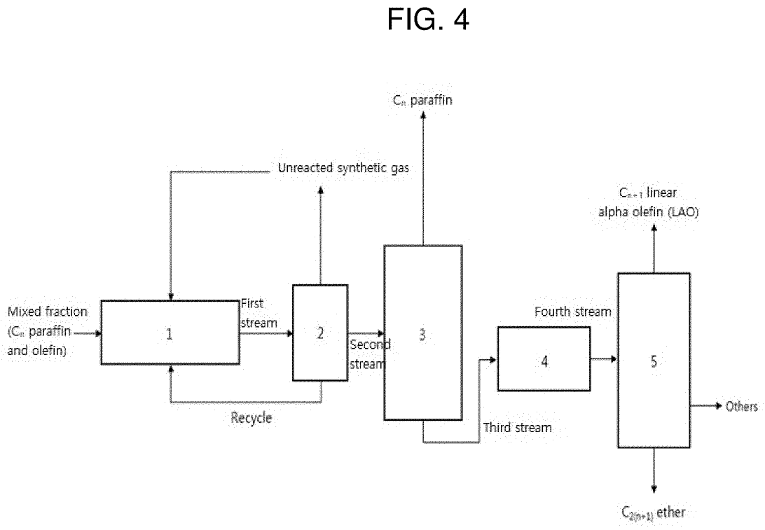

### DETAILED DESCRIPTION OF MAIN ELEMENTS

1: olefin-alcohol conversion reactor
2: solid-liquid separation apparatus
3: first distillation apparatus
4: dehydration apparatus
5: second distillation apparatus

### DETAILED DESCRIPTION OF EMBODIMENTS

Hereinafter, the method of preparing a linear primary alcohol according to the present invention will be described in detail with reference to the accompanying drawings.

The drawings illustrated in the present specification are provided by way of example so that the idea of the present invention may be sufficiently conveyed to a person skilled in the art. Therefore, the present invention is not limited to the provided drawings, but may be embodied in many different forms, and the drawings may be exaggerated in order to clear the spirit of the present invention.

Technical terms and scientific terms used in the present specification have the general meaning understood by those skilled in the art to which the present invention pertains unless otherwise defined, and a description for the known function and configuration which may unnecessarily obscure the gist of the present invention will be omitted in the following description and the accompanying drawings.

In addition, the singular form used in the specification of the present invention may be intended to also include a plural form, unless otherwise indicated in the context.

In addition, units used in the specification of the present invention without particular mention is based on weights, and as an example, a unit of % or ratio refers to a wt % or a weight ratio.

In addition, unless otherwise defined in the specification of the present invention, an average particle diameter refers to D₅₀ obtained by a particle size analyzer.

In addition, the numerical range used in the specification of the present invention includes all values within the range including the lower limit and the upper limit, increments logically derived in a form and span in a defined range, all double limited values, and all possible combinations of the upper limit and the lower limit in the numerical range defined in different forms. As an example, when it is defined that a content of a composition is 10% to 80%, specifically 20% to 50%, it should be interpreted as being that a numerical range of 10% to 50% or 50% to 80% is also described in the specification of the present invention. Unless otherwise particularly defined in the specification of the present invention, values which may be outside a numerical range due to experimental error or rounding of a value are also included in the defined numerical range.

In addition, in the specification of the present invention, the expression, “comprise” is an open-ended description having a meaning equivalent to the expression such as “is/are provided with”, “contain”, “have”, or “is/are characterized”, and does not exclude elements, materials, or processes which are not further listed. In addition, the expression, “substantially consisting of . . . ” means that other elements, materials, or processes which are not listed together with specified elements, materials, or processes may be present in an amount which does not have an unacceptable significant influence on at least one basic and novel technical idea of the invention. In addition, the expression, “consisting of” means that only the described elements, materials, or processes are present.

Various chemical byproducts are produced from processes such as Fisher-Tropsch synthesis or a naphtha cracker, and these byproducts include a mixed fraction containing a paraffin and an olefin. A hydroformylation process is used for preparing a linear α-olefin having a high added value from the mixed fraction, but since continuous production is difficult and a three or more-step process is included, a technique in which the process is further simplified, an operating condition of the process is more economical, and a linear α-olefin may be continuously produced with a high purity, should be established.

For solving the technical problems, the inventors of the present invention contrived development and process conditions of a heterogeneous cobalt oxide catalyst to develop a preparation method which may produce a linear primary alcohol with a high purity by a simpler process and economical operating conditions, thereby completing the present invention.

A method of preparing a linear primary alcohol according to the present invention includes charging a reactor with a heterogeneous catalyst including a cobalt oxide and a Cₙ (n is an integer of 4 to 20) olefin (S1); bringing the heterogeneous catalyst including a cobalt oxide into contact with the Cₙ olefin (S2); and supplying the reactor with a synthetic gas to obtain a Cₙ₊₁ alcohol (S3).

The method of preparing a linear primary alcohol according to the present invention may be performed batchwise, but more preferably, also has a merit of being performed continuously and various modified operation methods are also included in the scope of the present invention.

The heterogeneous catalyst including a cobalt oxide may be prepared by hydrothermal synthesis, using an aqueous solution of an aqueous cobalt salt as a starting material. Specifically, examples of the aqueous cobalt salt may include cobalt acetate, cobalt acetylacetonate, cobalt halide, cobalt nitrate, cobalt sulfate, and the like, and a solvate or hydrate thereof is also included in the scope of the present invention. The aqueous solution of the aqueous cobalt salt may be mixed with a surfactant, a reducing agent, or a combination thereof in a mixed solvent of water and an organic solvent and then be granulated by hydrothermal synthesis. The organic solvent used in the mixed solvent may be one or a combination of two selected from the group consisting of polar protic solvents, polar aprotic solvents, and the like. Here, examples of the polar protic solvent may include an aliphatic alcohol and the like, examples of the polar aprotic solvent may include alkyl formamide and the like, and the aliphatic alcohol and alkyl formamide may include an alkyl group having 1 to 7 carbon atoms. An example of the reducing agent may be urea, but is not limited thereto. In addition, the surfactant may be selected from a cationic surfactant selected from the group consisting of cetyltrimethylammonium bromide, cetyltrimethylammonium chloride, cetylpyridinium chloride, and the like; and a nonionic surfactant selected from the group consisting of polyvinylpyrrolidone (PVP), polyethylene glycol (PEG), polysorbate, and the like, but is not limited thereto.

Cobalt oxide particles obtained by hydrothermal synthesis may be subjected to a firing step subsequent to separation and washing steps, thereby being finally prepared into a cobalt oxide catalyst. The firing step may be performed in a temperature range of 200 to 600° C. for 1 hour to 8 hours, specifically in a temperature range of 300 to 500° C. for 2 hours to 6 hours, but is not limited thereto. The finally obtained cobalt oxide particles may be exemplified by a chemical formula of Co₃O₄.

The cobalt oxide heterogeneous particles may be easily recovered after completion of the reaction and be used, and is particularly advantageous for a continuous process, thereby improving process efficiency.

The cobalt oxide particles may have various shapes such as flake, flat plate, octahedral, spherical, rod, rectangular, hexagonal, and needle shapes, and the shapes mentioned above may be an example and the cobalt oxide particles are not limited to those shapes.

Preferably, the cobalt oxide particles may have a flat plate, octahedral, cube, spherical, or rod shape, and more preferably, may have a rod shape. As the cobalt oxide particles have a rod shape, a conversion rate of an olefin may be significantly increased and a selectivity of an alcohol when the olefin is converted into the alcohol may be also significantly increased, which is thus preferred.

When the cobalt oxide particles have a spherical shape, an average particle diameter thereof may be in a range of 10 to 100 nm, specifically in a range of 10 to 50 nm, and more specifically in a range of 20 to 40 nm.

In addition, when the cobalt oxide particles have a rod shape, the cross section thereof may have an average diameter in a range of 10 to 100 nm, specifically in a range of 10 to 50 nm, and an aspect ratio of 2 to 1000. More specifically, the average diameter may be 20 to 40 nm and the aspect ratio may be 5 to 500.

The Cₙ olefin refers to an olefin wherein a carbon number, n is an integer of 4 to 20, and specifically n may be 6 to 16, and may refer to a mixture of isomeric olefins as well as one olefin. For example, the Cₙ olefin may be preferably a monoolefin having a C═C double bond at a terminal position, and examples thereof may include 1-pentene, 1-hexene, 1-heptene, 1-octene, 1-nonene, and the like, but is not limited thereto. As a more specific example thereof, a paraffin-olefin mixed fraction including the Cₙ olefin may be charged into a reactor as a raw material of the olefin.

The synthetic gas includes carbon monoxide and hydrogen, and as an example, a mole ratio thereof may be 5:95 to 70:30, 40:80 to 60:40, or 1:2 to 1:1, and in this range, gas used in the reaction may not be accumulated in the reactor and a reaction balance may provide an excellent effect.

The above (S3) includes a reductive hydroformylation reaction of the Cₙ olefin and synthetic gas, and the synthetic gas and the Cₙ olefin are converted into a Cₙ₊₁ alcohol by a single reaction step through the reductive hydroformylation reaction. In an example of the present invention, the reductive hydroformylation reaction by the synthetic gas may partially remove risk factors from conventional use of hydrogen as a reactant at the time of a hydroformylation reaction and a hydrogenation reaction, and has a merit of preparing a higher alcohol by a single reaction step.

The reductive hydroformylation reaction of (S3) may be performed in a temperature range of 100° C. to 250° C., specifically in a range of 110° C. to 200° C. A reaction pressure may be 20 bar to 60 bar, specifically 30 bar to 50 bar.

In the reductive hydroformylation reaction, carbon monoxide and hydrogen interact with each other on a surface of the cobalt oxide particles to produce cobalt oxide particles which are surface-activated in the form of a carbonyl group, and an olefin molecule forms a composite with a cobalt metal of the cobalt oxide particles. The composite and the cobalt oxide particles which are surface-activated in the form of a carbonyl group are converted into a higher alcohol having increased carbon atoms by one on the surface of the cobalt oxide particles by a subsequent hydrogenation reaction.

In the reductive hydroformylation reaction, the synthetic gas is passed through a reaction mixture in which the Cₙ olefin and the heterogeneous catalyst are mixed by contact, by bubbling. By the bubbling of the synthetic gas, the reductive hydroformylation reaction is performed, and the product of the reaction forms a first stream. The first stream includes the heterogeneous catalyst, the Cₙ olefin, the Cₙ₊₁ alcohol, a trace amount of a Cₙ₊₁ aldehyde, and the synthetic gas which does not participate in the reaction. The synthetic gas which does not participate in the reaction may be separated as a gas from the first stream to be recycled to a reductive hydroformylation reactor.

The first stream from which the synthetic gas is separated is separated by a first catalyst removal step to remove the heterogeneous catalyst by a simple solid-liquid separation means, and the separated heterogeneous catalyst may be recycled to the reductive hydroformylation reactor or recycled after being supplemented with a newly prepared heterogeneous catalyst.

The Cₙ paraffin, the Cₙ₊₁ alcohol, and a trace amount of the Cₙ₊₁ aldehyde included in the first stream form a second stream, and the second stream is transferred to a distillation apparatus to be easily separated into the Cₙ₊₁ alcohol and the Cₙ paraffin using a difference in a boiling point.

As the distillation apparatus, a distillation apparatus known in the art may be used without limitation, and the Cₙ paraffin having a low boiling point is separated in an upper portion of the distillation apparatus and the Cₙ₊₁ alcohol having a high boiling point is separated in a lower portion of the distillation apparatus to form a third stream including the Cₙ₊₁ alcohol. The distillation apparatus may be operated under a reduced pressure condition, and for example, may be operated under a pressure of 400 to 900 mbar, but which is only an example, and the pressure is not limited thereto.

Selectively, the third stream including the Cₙ₊₁ alcohol may be purified by post-treatment into an alcohol having a higher purity by the distillation apparatus.

According to an exemplary embodiment of the present invention, a conversion rate of the olefin in (S3) may be 95% or more, and the selectivity of the Cₙ₊₁ alcohol may be 40% or more. Preferably, the conversion rate of the olefin may be 97.0% or more and the selectivity of the Cₙ₊₁ alcohol may be 40% or more, and more preferably, the conversion rate of the olefin may be 97.0% or more and the selectivity of the Cₙ₊₁ alcohol may be 45% or more.

The above steps of (S1) to (S3) do not necessarily mean sequential steps, and various modifications of steps are also included in the scope of the present invention. As a specific example, the steps may be performed in the manner that the synthetic gas is supplied into the reactor through a synthetic gas supply pipe, the reactor is charged with the heterogeneous catalyst including a cobalt oxide and the Cₙ olefin, and the heterogeneous catalyst and the Cₙ olefin are brought into contact with each other. As another specific example, the steps may be performed in the manner that the heterogeneous catalyst including a cobalt oxide and the Cₙ olefin are brought into contact with each other and then charged to the reactor, and the synthetic gas is supplied into the reactor through the synthetic gas supply pipe. Accordingly, the steps of (S1) to (S3) may be interpreted as one step, and an exemplary embodiment including the steps as one step is also included in the scope of the present invention.

According to an exemplary embodiment of the present invention, the cobalt oxide heterogeneous catalyst may be one or two or more metals selected from the group consisting of rhodium, palladium, silver, platinum, gold, iridium, ruthenium, and the like, and preferably, rhodium, silver, platinum, gold, or ruthenium supported on cobalt oxide particles. More preferably, the cobalt oxide heterogeneous catalyst particles may be rhodium, silver, platinum, gold, or ruthenium supported on rod-shaped cobalt oxide particles. More preferably, the cobalt oxide heterogeneous catalyst particles may be rhodium, platinum, or gold supported on rod-shaped cobalt oxide particles.

As the cobalt oxide heterogeneous catalyst has rhodium or ruthenium supported thereon, the selectivity of the Cₙ₊₁ alcohol may be 50% or more, preferably 60% or more. Here, the Cₙ₊₁ alcohol may have a structure of 1-alcohol, and may be obtained with a high selectivity.

The cobalt oxide heterogeneous catalyst having one or two or more metals selected from the group consisting of rhodium, palladium, silver, platinum, gold, iridium, ruthenium, and the like supported thereon may be prepared by hydrothermal synthesis using an aqueous cobalt salt precursor and an aqueous metal salt precursor of the metal as starting materials. Specifically, the cobalt oxide heterogeneous catalyst may be prepared through granulation by preparing a first precursor solution in which the aqueous cobalt precursor is dissolved in a mixed solvent in which water and a polar protic solvent or a surfactant are further mixed; mixing the first precursor solution with a base solution to induce precipitation; mixing an aqueous metal salt of one or two or more metals selected from the group consisting of rhodium, palladium, silver, platinum, gold, iridium, ruthenium, and the like with water and a reducing agent to prepare a second precursor solution; adding the second precursor solution dropwise to the first precursor solution and mixing them; and hydrothermally synthesizing the mixed solution. The polar protic solvent may be an aliphatic alcohol, and an example of the reducing agent may be urea, but they are not limited thereto.

The cobalt oxide particles having one or two or more metals selected from the group consisting of rhodium, palladium, silver, platinum, gold, iridium, ruthenium, and the like supported thereon, obtained by hydrothermal synthesis may be subjected to a firing step subsequent to a separation and washing step to be finally prepared into a cobalt oxide catalyst having one or two or more metals selected from the group consisting of rhodium, palladium, silver, platinum, gold, iridium, ruthenium, and the like supported thereon.

According to an exemplary embodiment of the present invention, after (S3), dehydrating the Cₙ₊₁ alcohol to obtain a Cₙ₊₁ olefin (S4) may be further included. In (S4), the Cₙ₊₁ alcohol obtained in (S3) is dehydrated to be converted into the Cₙ₊₁ olefin. As described above, the third stream including the Cₙ₊₁ alcohol separated from the distillation apparatus is dehydrated in (S4), and the dehydration reaction in (S4) is performed in a dehydration reactor. More specifically, the dehydration reactor is provided with an alumina catalyst, and as a specific example, a column filled with γ-alumina catalyst is included.

A dehydration reaction of an alcohol may be performed by passing the alcohol through an alumina-filled layer in a temperature range of 150° C. to 400° C., and as the specific dehydration operating conditions through the alumina-filled column, conditions known in the art may be used without limitation.

The third stream is passed through the alumina catalyst and is converted into the Cₙ₊₁ olefin by a dehydration reaction to finally obtain the Cₙ₊₁ linear α-olefin. Since a mixture including the Cₙ₊₁ linear α-olefin, a small amount of a Cₙ₍ₙ₊₁₎ ether, and byproducts may be produced as a main product by the dehydration reaction, the mixture subjected to the dehydration reaction forms a fourth stream and may be transferred to the distillation apparatus.

As the distillation apparatus, a distillation apparatus known in the art may be used without limitation, and the Cₙ₊₁ olefin having a low boiling point, that is, the Cₙ₊₁ linear α-olefin is separated from the upper portion of the distillation apparatus and recovered, and a C₂₍ₙ₊₁₎ ether having a high boiling point is separated from the lower portion of the distillation apparatus and recovered. As a specific example, when the Cₙ₊₁ linear α-olefin is 1-octene, the boiling point thereof is 121° C., and the C₂₍ₙ₊₁₎ ether is dioctyl ether, the boiling point thereof is 286° C., and thus, it is possible to recover 1-octene easily with a high purity by simple distillation.

The distillation apparatus may be operated under a reduced condition. The Cₙ₊₁ linear α-olefin having a low boiling point may be recovered with a high purity by distillation, and the purity may be 96% or more, more specifically 99% or more.

In addition, the present invention provides a catalyst for converting an α-olefin into an alcohol, and the catalyst for converting an α-olefin into an alcohol according to the present invention includes a heterogeneous catalyst including a cobalt oxide and may convert a Cₙ olefin into a Cₙ₊₁ alcohol with high selectivity and conversion rate. Preferably, the catalyst for converting an α-olefin into an alcohol may be a heterogeneous catalyst including a spherical or rod-shaped cobalt oxide, and more preferably, the catalyst for converting an α-olefin into an alcohol may be the heterogeneous catalyst including a rod-shaped cobalt oxide.

As a preferred example of the catalyst for converting an α-olefin into an alcohol according to the present invention, the heterogenous catalyst may be one or two or more metals selected from the group consisting of rhodium, palladium, platinum, silver, gold, iridium, ruthenium, and the like supported on cobalt oxide particles, and a more preferred example thereof may be rhodium, silver, platinum, gold, ruthenium, or a combination thereof supported on cobalt oxide particles. The cobalt oxide and one or two or more metals selected from the group consisting of rhodium, silver, platinum, gold, or ruthenium may be included at a weight ratio of 1:0.001 to 1:0.1. In addition, as a preferred example, the metal may be included at 0.01 to 5 wt %, and as a more preferred example, may be included at 0.1 to 3 wt %, based on a total weight of the catalyst for converting an α-olefin into an alcohol.

By the heterogeneous catalyst for converting an α-olefin into an alcohol as described above, the present invention provides a method of bringing a mixed fraction including a paraffin and a Cₙ olefin into contact with the heterogeneous catalyst to convert the mixed fraction into a Cₙ₊₁ alcohol by a reductive hydroformylation reaction. That is, the olefin from a paraffin-olefin mixed fraction having n carbon atoms produced from various chemical byproducts such as chemical byproducts from Fisher-Tropsch synthesis or a naphtha cracker may be selectively converted into the alcohol having n+1 carbon atoms, and the alcohol and the olefin may be easily separated using a difference in a boiling point. In addition, the separated alcohol is transferred to a continuous reactor, and then is converted into a linear α-olefin having n+1 carbon atoms by a dehydration reaction to produce a high-purity linear α-olefin.

A traditional hydroformylation method necessarily includes a hydrogenation process and needs a two or more-step process, and as an unreacted olefin is hydrogenated at the time of the hydrogenation process, the content of an inactive saturated hydrocarbon, that is, the paraffin, is increased to decrease an alcohol conversion rate of the olefin, resulting in a decrease of the content of the olefin to be recycled. However, the method of converting a Cₙ₊₁ alcohol according to the present invention converts the olefin into the alcohol by a one-step process by the reductive hydroformylation reaction, thereby having merits of inhibiting the olefin from being converted into the paraffin and converting the olefin into the alcohol with a high yield, and may also simplify a separation process.

Hereinafter, the present invention will be described in detail by the Examples, however, the Examples are for describing the present invention in more detail, and the scope of the present invention is not limited to the following Examples.

### [Preparation Example 1] Preparation of Flake-Shaped Heterogeneous Cobalt Oxide (Co₃O₄) Catalyst

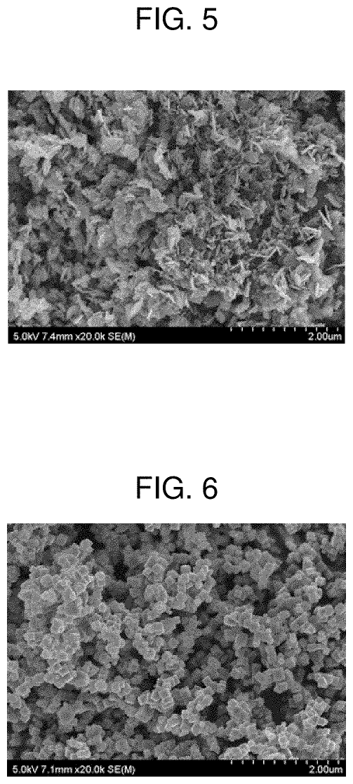

In one beaker (A), 100 ml of an aqueous cobalt nitrate hexahydrate solution having a concentration of 0.1 M was prepared, and in the other beaker (B), 200 ml of an aqueous solution in which polyvinylpyrrolidone (PVP), ethanol, and water were mixed (PVP:ethanol:water=1:10:1, weight ratio) was prepared. A caustic soda (NaOH) solution having a concentration of 0.11 M was slowly added dropwise to a mixed solution of (A) and (B) while stirring the solution with a magnetic bar to induce precipitation, and then the solution was placed in a hydrothermal synthesis reactor coated with Teflon and was reacted at 120° C. for 10 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a cobalt oxide catalyst. The electron microphotograph of the obtained flake-shaped heterogeneous cobalt oxide is illustrated in FIG. 5.

### [Preparation Example 2] Preparation of Cube-Shaped Heterogeneous Cobalt Oxide (Co₃O₄) Catalyst

In one beaker (A), 100 ml of an aqueous cobalt nitrate hexahydrate solution having a concentration of 0.1 M was prepared, and in the other beaker (B), a caustic soda (NaOH) solution having a concentration of 0.1 M was prepared. The solution (B) was added dropwise to the solution (A) while slowly stirring the solution with a magnetic bar to induce precipitation, and then the solution was placed in a hydrothermal synthesis reactor coated with Teflon and was reacted at 120° C. for 10 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a cobalt oxide catalyst. The electron microphotograph of the obtained cube-shaped heterogeneous cobalt oxide is illustrated in FIG. 6.

### [Preparation Example 3] Preparation of Spherical Heterogeneous Cobalt Oxide (Co₃O₄) Catalyst

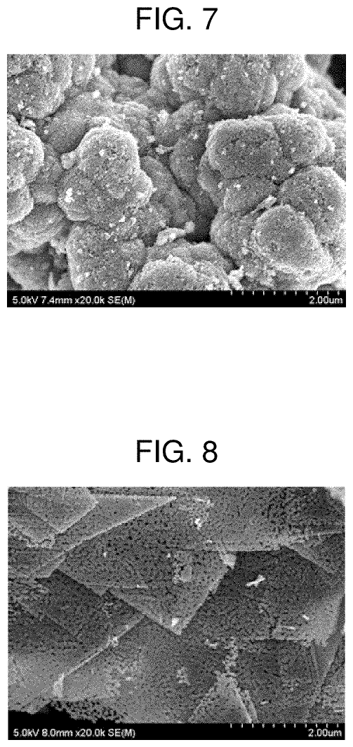

In one beaker (A), 100 ml of an aqueous cobalt acetate tetrahydrate solution having a concentration of 0.1 M was prepared, and in the other beaker (B), an ethylene glycol solution having a concentration of 0.1 M was prepared. The solution (B) was added dropwise to the solution (A) while slowly stirring the solution with a magnetic bar, and then the solution was placed in a hydrothermal synthesis reactor coated with Teflon and was reacted at 200° C. for 6 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a cobalt oxide catalyst. The electron microphotograph of the obtained spherical heterogeneous cobalt oxide is illustrated in FIG. 7.

### [Preparation Example 4] Preparation of Flat Plate-Shaped Heterogeneous Cobalt Oxide (Co₃O₄) Catalyst

In one beaker (A), 100 ml of an aqueous cobalt chloride hexahydrate solution having a concentration of 0.1 M was prepared, and in the other beaker (B), a caustic soda solution having a concentration of 0.1 M was prepared. The solution (B) was added dropwise to the solution (A) while slowly stirring the solution with a magnetic bar to induce precipitation, and then the solution was placed in a hydrothermal synthesis reactor coated with Teflon and was reacted at 200° C. for 6 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a cobalt oxide catalyst. The electron microphotograph of the obtained flat plate-shaped heterogeneous cobalt oxide is illustrated in FIG. 8.

### [Preparation Example 5] Preparation of Octahedral Heterogeneous Cobalt Oxide (Co₃O₄) Catalyst

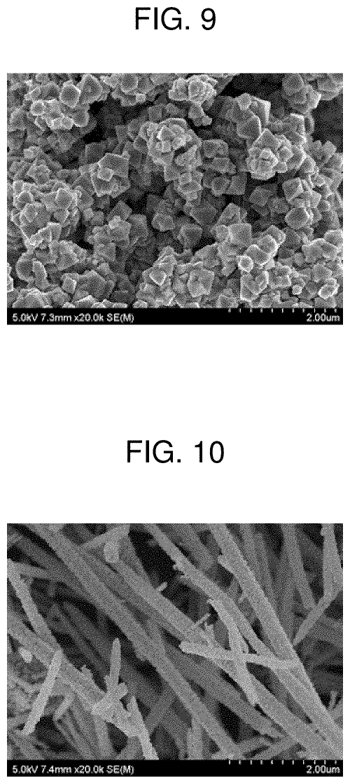

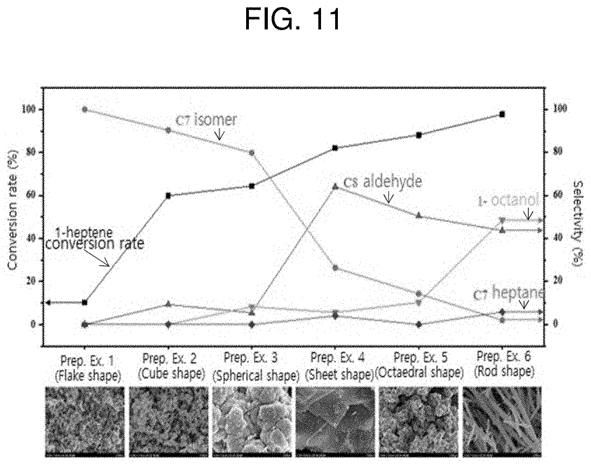

In one beaker (A), a 0.2 M aqueous cobalt chloride hexahydrate solution was prepared, and in the other beaker (B), a urea solution having a concentration of 0.4 M was prepared. The solution (A) and the solution (B) were slowly added dropwise to 100 ml of distilled water using a magnetic bar to induce precipitation, and the solution was allowed to stand for 6 hours or more. The solution was placed in a hydrothermal synthesis reactor coated with Teflon and was reacted at 200° C. for 16 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a octahedral heterogeneous cobalt oxide catalyst. The electron microphotograph of the obtained octahedral heterogeneous cobalt oxide is illustrated in FIG. 9.

### [Preparation Example 6] Preparation of Rod-Shaped Heterogeneous Cobalt Oxide (Co₃O₄) Catalyst

In one beaker (A), 100 mg of a cobalt chloride hexahydrate precursor was sufficiently mixed with 300 ml of a mixed solution in which cetyltrimethylammonium bromide (CTAB) which is a cationic surfactant and water were mixed at a weight ratio of 1:3, the prepared solution was slowly added dropwise to the other beaker (B) including 50 ml of a 1N aqueous NaOH solution while stirring the solution, and the solution in the beaker (B) was allowed to stand for 6 hours or more. A solution having a concentration of 0.5 M in which urea is dissolved in water was slowly added dropwise to the above solution while stirring the solution, and was placed in a hydrothermal synthesis reactor coated with Teflon and was reacted at 120° C. for 12 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a rod-shaped cobalt oxide catalyst. The electron microphotograph of the obtained rod-shaped heterogeneous cobalt oxide is illustrated in FIG. 10.

### [Preparation Example 7] Platinum (Pt) Catalyst Supported on Rod-Shaped Heterogeneous Cobalt Oxide (Co₃O₄)

In one beaker (A), 100 mg of a cobalt chloride hexahydrate precursor was sufficiently mixed with 300 ml of a mixed solution in which cetyltrimethylammonium bromide (CTAB) which is a cationic surfactant and water were mixed at a weight ratio of 1:3, the prepared solution was slowly added dropwise to the other beaker (B) including 50 ml of a 1N aqueous NaOH solution, and the solution in the beaker (B) was allowed to stand for 6 hours or more. A solution in which urea and platinum tetrachloride were dissolved in water at concentrations of 0.5 M and 0.1 M, respectively to be mixed, was slowly added dropwise to the above solution while stirring the solution, and the solution was placed in a hydrothermal synthesis reactor coated with Teflon and was reacted at 120° C. for 12 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a rod-shaped cobalt oxide catalyst having platinum supported thereon.

### [Preparation Example 8] Rhodium (Rh) Catalyst Supported on Rod-Shaped Heterogeneous Cobalt Oxide (Co₃O₄)

In one beaker (A), 100 mg of a cobalt chloride hexahydrate precursor was sufficiently mixed with 300 ml of a mixed solution in which cetyltrimethylammonium bromide (CTAB) which is a cationic surfactant and water were mixed at a weight ratio of 1:3, the prepared solution was slowly added dropwise to the other beaker (B) including 50 ml of a 1N aqueous NaOH solution, and the solution in the beaker (B) was allowed to stand for 6 hours or more. A solution in which urea and rhodium chloride were dissolved in water at concentrations of 0.5 M and 0.1 M, respectively to be mixed was slowly added dropwise to the above solution while stirring the solution, and the solution was placed in a hydrothermal synthesis reactor coated with Teflon and was reacted at 120° C. for 12 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a rod-shaped cobalt oxide catalyst having rhodium supported thereon.

### [Preparation Example 9] Gold (Au) Catalyst Supported on Rod-Shaped Heterogeneous Cobalt Oxide (Co₃O₄)

In one beaker (A), 100 mg of a cobalt chloride hexahydrate precursor was sufficiently mixed with 300 ml of a mixed solution in which cetyltrimethylammonium bromide (CTAB) which is a cationic surfactant and water were mixed at a weight ratio of 1:3, the prepared solution was slowly added dropwise to the other beaker (B) including 50 ml of a 1N aqueous NaOH solution, and the solution in the beaker (B) was allowed to stand for 6 hours or more. A solution in which urea and gold chloride hydrate were dissolved in water at concentrations of 0.5 M and 0.1 M, respectively to be mixed was slowly added dropwise to the above solution while stirring the solution, and the solution was placed in a hydrothermal synthesis reactor coated with Teflon and was reacted at 120° C. for 12 hours. After the reaction, the mixture taken out was washed twice or more with water, washed again three times with ethanol, and then was sufficiently dried using a vacuum oven at 40° C. The obtained product was fired at 500° C. for 4 hours in an air atmosphere to obtain a rod-shaped cobalt oxide catalyst having gold supported thereon.

### Examples 1 to 6

Compositions and boiling points of the paraffin-olefin mixed fractions used for catalyst performance evaluation are shown in the following Table 1.

TABLE 1

| | | Boiling |
|---|---|---|
| Components | Composition (v/v %) | point (° C.) |
| n-Heptane (C7 paraffin) | 50.0 | 98.4 |
| 1-Heptene (C7 olefin) | 40.7 | 93.6 |
| 2-Heptene (C7 olefin) | 3.2 | 98.5 |
| 2-methyl-1-hexene (C7 olefin) | 6.1 | 90.9 |

The reaction experiment of the first step was performed by the following method. A reactor was charged with 100 ml of a reactant and 0.1 g of each of the catalysts prepared in Preparation Examples 1 to 6 under a nitrogen atmosphere in a glove box, and was connected to a synthetic gas line in which a ratio of hydrogen and carbon monoxide was 1:1 by volume.

A reaction temperature was set at 170° C. and an operating pressure was set as 45 bar, and the reaction was performed by operation for 12 hours. The product obtained by the reaction was analyzed using gas chromatography with an FID detector. Conversion rates and selectivity of the olefin are shown in Table 2, and each of the conversion rates and selectivity was calculated by the following manner. The test results by the catalysts are indicated as the conversion rates and the selectivity of the olefin in Table 2.

$${c}_{7 } olefin conversion rate ( \% ) = \frac{( \begin{matrix}Moles of olefin reactant - \\ Moles of olefin in product\end{matrix} )}{Moles of olefin in reactant} \times 100$$

$${C}_{8} aldehyde selectivity ( \% ) = \frac{Moles of {C}_{8} aldehyde in product}{Moles of converted olefin} \times 100$$

$$1 - octanol selectivity ( \% ) = \frac{Moles of 1 - octanol in product}{Moles of converted olefin} \times 100$$

### Examples 7 to 9

The reaction experiment of the first step was performed in a similar manner to Example 1. Specifically, a reactor was charged with 100 ml of a reactant and 0.1 g of each of the catalysts prepared in Preparation Examples 7 to 9 under a nitrogen atmosphere in a glove box, and was connected to a synthetic gas line which was set as the volume ratios shown in the following Table 2.

After the reaction temperature and the operating pressure shown in the following Table 2 were set, the reaction was performed by operation for 20 hours. The product obtained by the reaction was analyzed using gas chromatography with an FID detector. The test results by the catalysts are indicated as the conversion rates and the selectivity of the olefin in the following Table 2. In addition, in the present Example, in order to confirm that the selectivity of a C₈ alcohol is improved, the reaction temperature was adjusted to 130° C. Specifically, in the present Example, when the reaction was performed at 170° C., it was confirmed that the olefin conversion rate of 100% was shown in all of the cases of Examples 7 to 9.

### Example 10

The alcohol product obtained in Example 6 was treated at 80° C. for about 10 minutes in a reduced pressure state in a rotary evaporative concentrator and, the compositions and the boiling points of a fraction (A) obtained from volatilization and a fraction (B) which remained after volatilization are shown in the following Table 3.

Subsequently, a dehydration reaction as the second step was performed using the residual fraction (B) as a reactant. The vaporized reactant was passed through a catalyst layer while the residual fraction (B) was supplied by a pump to a preheater together with nitrogen at a speed of 0.05 ml/min, thereby performing a dehydration reaction. The temperatures of the preheater and the reactant were maintained at 280° C. and 350° C., respectively, and as the reactor, a ⅜ inch quartz reactor was loaded with 0.2 g of γ-alumina catalyst. The compositions before and after the reaction are shown in the following Table 4.

### Comparative Example 1

Tris(triphenylphosphine)rhodium carbonyl hydride (Sigma-Aldrich) which is a homogeneous precious metal catalyst was used as a catalyst for a hydroformylation reaction.

In order to evaluate the performance of the homogeneous precious metal catalyst, the reaction experiment of the first step was performed by the following method. A reactor was charged with 100 ml of the reactant and 0.2 mg of the tris(triphenylphosphine)rhodium carbonyl hydride catalyst under a nitrogen atmosphere in a glove box, and was connected to a synthetic gas line in which a ratio of hydrogen and carbon monoxide was 1:1 by volume.

After a reaction temperature was set at 170° C. and an operating pressure was set as 20 bar, the reaction was performed by operation for 12 hours. The product obtained by the reaction was analyzed using gas chromatography with an FID detector. The test results by the catalysts are indicated as the conversion rates and the selectivity of the olefin in the following Table 2.

### Comparative Example 2

Dicobalt octacarbonyl (Sigma-Aldrich) which is a homogeneous transition metal catalyst was subjected to pretreatment under a hydrogen atmosphere, and was used as a catalyst for the hydroformylation reaction.

In order to evaluate the performance of the homogeneous transition metal catalyst, the reaction experiment of the first step was performed by the following method. A reactor was charged with 100 ml of the reactant and 0.2 mg of the dicobalt octacarbonyl catalyst under a nitrogen atmosphere in a glove box, and was connected to a synthetic gas line in which a ratio of hydrogen and carbon monoxide was 1:1 by volume.

Since the transition metal catalyst requires a higher reaction temperature and a higher pressure than the precious metal catalyst, the reaction temperature was set at 250° C. and the operating pressure was set at 40 bar, and the reaction was performed by operation for 12 hours. The product obtained by the reaction was analyzed using gas chromatography with an FID detector. The test results by the catalysts are indicated as the conversion rates and the selectivity of the olefin in the following Table 2.

TABLE 2

| | | | | | C₇ olefin | C₈ | 1- |
|---|---|---|---|---|---|---|---|
| | | Reaction | | Reaction | conversion | aldehyde | octanol |
| Reaction | Catalyst | temperature | CO/H₂ | pressure | rate | selectivity | selectivity |
| example | used | (° C.) | ratio | (bar) | (%) | (%) | (%) |
| Comparative | A-1 | 120 | 1:1 | 20 | 91 | 96.9 | 1.2 |
| Example 1 | | | | | | | |
| Comparative | A-2 | 250 | 1:1 | 40 | 65 | 76.4 | 0 |
| Example 2 | | | | | | | |
| Example 1 | Preparation | 170 | 1:1 | 45 | 10.2 | 0 | 0 |
| | Example 1 | | | | | | |
| Example 2 | Preparation | 170 | 1:1 | 45 | 59.9 | 9.3 | 0 |
| | Example 2 | | | | | | |
| Example 3 | Preparation | 170 | 1:1 | 45 | 64.4 | 5.4 | 8.3 |
| | Example 3 | | | | | | |
| Example 4 | Preparation | 170 | 1:1 | 45 | 82.0 | 64.1 | 5.6 |
| | Example 4 | | | | | | |
| Example 5 | Preparation | 170 | 1:1 | 45 | 88.1 | 50.4 | 10.3 |
| | Example 5 | | | | | | |
| Example 6 | Preparation | 170 | 1:1 | 45 | 97.7 | 43.7 | 48.5 |
| | Example 6 | | | | | | |
| Example 7 | Preparation | 130 | 1:1 | 45 | 72.9 | 2.4 | 70.7 |
| | Example 7 | | | | | | |
| Example 8 | Preparation | 130 | 1:1 | 45 | 80.7 | 11.8 | 69.2 |
| | Example 8 | | | | | | |
| Example 9 | Preparation | 130 | 1:1 | 45 | 63.6 | 0.6 | 63.1 |
| | Example 9 | | | | | | |

Comparative Example 1 (A-1): tris(triphenylphosphine)rhodium carbonyl hydride, which is a homogeneous precious metal catalyst, was used.
Comparative Example 2 (A-2): dicobalt octacarbonyl which is a homogeneous transition metal catalyst was pretreated under a hydrogen atmosphere and then used.

TABLE 3

| | Boiling | Volatilized | Residual |
|---|---|---|---|
| Components | point (° C.) | fraction-A (%) | fraction-B (%) |
| n-Heptane (C7 paraffin) | 98.4 | 97 | 0.1 |
| 1-Heptene (C7 olefin) | 93.6 | — | — |
| 2-Heptene (C7 olefin) | 98.5 | — | — |
| 2-methyl-1-hexene (C7 | 90.9 | 0.9 | — |
| olefin) | | | |
| Octanal (C8 aldehyde) | 171 | — | 2.2 |
| 1-Octanol (C8 alcohol) | 188 | — | 96 |
| Others | — | 2.1 | 1.7 |

TABLE 4

| Components | Before reaction (%) | After reaction (%) |
|---|---|---|
| 1-Octanol (C8 alcohol) | 96 | 0.9 |
| Others (C8 Oxygenates) | 4 | |
| Dioctyl ether (C16 high | — | 19.1 |
| value added product) | | |
| 1-Octene (C8 α-olefin) | — | 80 |

The method of preparing a linear primary alcohol according to the present invention may selectively convert a Cₙ olefin from a paraffin-olefin mixed fraction produced from various chemical byproducts into a Cₙ₊₁ alcohol with a high purity, and simplify a two or more-step process into a one-step process simultaneously with economically providing a Cₙ₊₁ linear primary alcohol under a low temperature and low pressure condition.

In addition, the cobalt oxide heterogeneous catalyst according to the present invention has merits of higher process efficiency than a conventional homogeneous catalyst and being capable of continuous treatment, at the time of conversion an α-olefin into an alcohol.

The effects described in the specification which is expected by the technical features of the present invention and the intrinsic effects are regarded as being described in the specification of the present invention, though the effects are not explicitly mentioned in the present invention.

What is claimed is:

1. A method of preparing a linear primary alcohol, the method comprising:
charging a reactor with a heterogeneous catalyst including a cobalt oxide and a Cₙ olefin (S1);
bringing the heterogeneous catalyst including a cobalt oxide into contact with the Cₙ olefin (S2); and
supplying the reactor with a synthetic gas to obtain a Cₙ₊₁ alcohol (S3),
wherein n is an integer of 4 to 20.

2. The method of preparing a linear primary alcohol of claim 1, wherein (S3) includes a reductive hydroformylation reaction of the Cₙ olefin and the synthetic gas.

3. The method of preparing a linear primary alcohol of claim 2, wherein the reductive hydroformylation reaction is performed at a temperature of 100° C. to 350° C. under a pressure of 15 bar to 60 bar.

4. The method of preparing a linear primary alcohol of claim 1, wherein the cobalt oxide has a rod shape.

5. The method of preparing a linear primary alcohol of claim 4, wherein a cross section of the rod-shaped cobalt oxide has an average diameter of 10 to 100 nm and an aspect ratio of 2 to 1000.

6. The method of preparing a linear primary alcohol of claim 1, wherein the heterogeneous catalyst is one or two or more metals selected from the group consisting of rhodium, palladium, platinum, silver, gold, iridium, and ruthenium supported on cobalt oxide particles.

7. The method of preparing a linear primary alcohol of claim 1, wherein a conversion rate of the olefin in (S3) is 95% or more.

8. The method of preparing a linear primary alcohol of claim 1, wherein a selectivity of the Cₙ₊₁ alcohol in (S3) is 40% or more.

9. The method of preparing a linear primary alcohol of claim 1, further comprising: after (S3), dehydrating the Cₙ₊₁ alcohol to obtain a Cₙ₊₁ olefin (S4).

10. The method of preparing a linear primary alcohol of claim 9, wherein (S4) includes the dehydrating under an alumina catalyst.

11. A catalyst for converting an α-olefin into a primary alcohol, comprising a heterogeneous catalyst including a cobalt oxide, which is used for converting a Cₙ olefin into a Cₙ₊₁ alcohol.

12. The catalyst for converting an α-olefin into a primary alcohol of claim 11, wherein the heterogeneous catalyst is one or two or more metals selected from the group consisting of rhodium, palladium, platinum, silver, gold, iridium, and ruthenium supported on cobalt oxide particles.

13. The catalyst for converting an α-olefin into a primary alcohol of claim 12, wherein the cobalt oxide and the supported metal are included at a weight ratio of 1:0.001 to 1:0.1.

14. A method of converting an olefin into an alcohol, the method comprising: bringing a mixed fraction including a paraffin and a Cₙ olefin into contact with a heterogeneous catalyst including a cobalt oxide to convert the mixed fraction into a Cₙ₊₁ primary alcohol by a reductive hydroformylation reaction.
